(19) United States
(12) Patent Application Publication
(10) Pub. No.: US 2017/0367463 A1
(43) Pub. Date: Dec. 28, 2017

(54) COSMETIC APPLICATOR HAVING AN INTERNAL SPACE CONTAINING THE COMPOSITION

(71) Applicant: L'OREAL, Paris (FR)

(72) Inventor: Alexis LEONARD, Clichy (FR)

(73) Assignee: L'OREAL, Paris (FR)

(21) Appl. No.: 15/542,034

(22) PCT Filed: Jan. 5, 2016
(86) PCT No.: PCT/EP2016/050094

§ 371 Date: Jul. 6, 2017

(30) Foreign Application Priority Data
Jan. 6, 2015 (FR) 15 50076

(51) Int. Cl. A45D 40/26; A61K 8/88; A61K 8/02; A61Q 1/10

(52) U.S. Cl. CPC A45D 40/262; A61Q 1/10; A61K 8/88; A61K 8/0254; A61K 8/027; A61K 2800/87

(57) ABSTRACT

The present invention relates to a cosmetic applicator for applying a non-liquid composition (P) to human keratin materials, the applicator: —comprising a stem (7) that extends along a longitudinal axis (X), —defining an internal space (9) that is able to contain the composition (P) to be applied, and —having at least one opening (18) that is in communication with the internal space (9) and leads onto an application surface (8).

The present invention relates to applicators for applying a cosmetic, makeup or care, product to human keratin materials, and to packaging and application devices comprising the applicator and a container.

The applicators known for applying a composition to human keratin materials, in particular mascara to the eyelashes and/or eyebrows, a lip gloss or a liquid eyeshadow, are often intended to be dipped into a container containing the composition to be applied, this container generally comprising a wiping member.

These application devices are not very suitable for dispensing and applying non-liquid compositions, in particular compositions which are pulverulent, pasty and/or comprise solid particles, such as fibres or flakes, since it is difficult to precisely load the applicator member with such compositions.

It is furthermore possible for the applicator members of these devices not to be suited to applying such compositions, for example not making it possible to retain sufficient solid particles, which tend to come off the applicator on passing through the wiping member.

Applications EP 1 264 562 and FR 2 976 165 describe applicator devices intended for applying a composition comprising fibres.

U.S. Pat. No. 5,547,302 describes a composition applicator in the form of a stick with a driving wheel, comprising a mesh applicator head.

There is a need to further improve applicators for applying a composition which is pulverulent, pasty or comprises fibres or flakes to human keratin materials, so as to improve the performance thereof, and in particular to improve the dispensing, quantity and distribution of the composition on the application surface of the applicator.

The invention aims to meet this objective and the subject thereof, according to one of its aspects, is a cosmetic applicator for applying a non-liquid composition to human keratin materials, the applicator
comprising a stem that extends along a longitudinal axis,
defining an internal space that is able to contain the composition to be applied, and
having at least one opening that is in communication with the internal space and leads onto an application surface.

A further subject of the invention, according to another of its aspects, is a cosmetic applicator for applying a composition to human keratin materials, the applicator:
comprising a stem that extends along a longitudinal axis and bears an endless screw,
defining an internal space that is able to contain the composition to be applied, and
having at least one opening that is in communication with the internal space in order to dispense said composition,
   wherein the endless screw turns inside the internal space such that its rotation causes the composition to flow through the opening(s).

By virtue of the invention, the composition is delivered in a controlled manner, the user being able to easily meter the quantity of composition distributed through the opening(s) of the applicator, by shaking it more or less or by turning the endless screw.

Moreover, in order to load the application surface with composition, it is not necessary to dip the applicator into a container containing the composition, thereby making it possible to keep it relatively clean.

Internal Space and Openings

The wall of the applicator having the opening(s) advantageously defines the application surface for the composition. In a preferred embodiment, the stem has the opening(s) close to its distal end, and defines said application surface.

The internal space is advantageously defined by the body of the stem, preferably extending as far as the distal end thereof.

The internal space preferably contains the non-liquid composition to be applied.

The ratio between the volume taken up by the composition within the internal space and the volume of the latter is preferably between ⅓ and 1, better still between ⅖ and ⅘.

The applicator advantageously has a plurality of openings in the form of slots, each preferably extending along a longitudinal axis perpendicular to the longitudinal axis of the stem. In one variant, the openings each extend along an axis that is not perpendicular to the longitudinal axis of the stem, for example at an angle or parallel thereto. In another variant, the openings are orifices with some other shape, in particular with a circular or square section.

Openings in the form of perpendicular slots provide the user with a combing effect, this being particularly advantageous when the composition is applied to the eyelashes, the eyebrows or the hair.

The applicator can have between 5 and 50 openings, better still between 10 and 20 openings.

Each opening can be formed over an angular sector of between 90° and 300° about the longitudinal axis of the stem, in particular over an angular sector equal to 180°. The openings advantageously extend to half the thickness of the stem of the applicator, when seen in side view, and across the entire width thereof, when seen in front view.

The most distal opening can extend to a non-zero distance from the distal end of the applicator, this distance being measured along the longitudinal axis of the stem, in particular between 2 mm and 5 mm, better still between 3 mm and 4 mm.

The spacing between the openings, measured along the longitudinal axis of the stem, may be between 0.3 mm and 5 mm, better still between 0.4 mm and 1.5 mm.

The width of the openings, measured perpendicularly to their longitudinal axis, may be between 0.5 mm and 3 mm, better still between 0.8 mm and 1.4 mm.

The depth of the openings, measured perpendicularly to their longitudinal axis when the applicator is seen from the side, may be between 1.9 mm and 4 mm, better still between 2.5 mm and 3.5 mm.

The number and dimensions of the openings, and the spacing thereof, may vary depending on the composition to be applied.

Endless Screw

The endless screw can extend along a rectilinear longitudinal axis, in particular coincident with the longitudinal axis of the stem.

When the internal space is defined by the entire length of the body of the stem, the endless screw advantageously extends along substantially the entire length of the stem. In one variant, the endless screw is shorter than the stem.

The thread of the endless screw is preferably continuous. In one variant, the thread of the endless screw is discontinuous.

The width of the thread of the endless screw, measured perpendicularly to the thread, at its base, may be between 0.5 mm and 1.3 mm, better still between 0.7 mm and 1 mm. The pitch of the endless screw is preferably between 4 mm and 8 mm, better still between 5 mm and 7 mm.

The endless screw advantageously comprises a body and protruding elements, such as spikes, that are connected thereto and make it easier to dispense the composition. The protruding elements of the endless screw make it possible to scrape and decompact the composition, in particular the solid particles when it comprises them.

The endless screw may comprise between 5 and 200 protruding elements, better still between 50 and 80 protruding elements, in particular between 1 and 10 protruding elements per turn.

These protruding elements are advantageously disposed along grooves formed by the thread of the endless screw. The protruding elements advantageously follow the same helical path as the thread of the latter.

The protruding elements are advantageously the same height as the thread. In one variant, the protruding elements have a smaller or greater height than the thread of the endless screw.

The height of the thread of the endless screw, and of the protruding elements if need be, measured perpendicularly to the longitudinal axis of the endless screw, may be between 0.5 mm and 1.5 mm, better still between 0.7 mm and 1.2 mm.

The largest transverse dimension of the protruding elements of the endless screw, measured at their base, may be between 0.5 mm and 1.4 mm, better still between 0.7 mm and 1.1 mm.

The cross section of the body of the endless screw may be variable along the longitudinal axis of the stem. The endless screw may have a narrowing in its cross section, in particular situated closer to its proximal end than to its distal end.

The diameter of the body of the endless screw, over the proximal portion of the screw situated upstream of the narrowing, starting from the proximal end, may be between 5 mm and 10 mm. The diameter of the body of the endless screw, over the distal portion of the screw situated downstream of the narrowing, starting from the proximal end, may be between 1.5 mm and 4 mm.

The clearance between the stem and the thread of the endless screw may be between 0.1 mm and 4 mm, better still between 0.5 mm and 3 mm. This clearance may be constant along the longitudinal axis of the stem. In one variant, the clearance is variable along the longitudinal axis of the stem, reaching for example a maximum over the proximal portion of the stem situated upstream of the narrowing.

The body of the endless screw may be hollow along at least a part of its length, in particular over its proximal portion upstream of the narrowing.

The endless screw may be immobilized axially by any means on the stem, in particular with the aid of a shoulder in its upper part, while being able to be rotated. The endless screw may comprise an annular bulge in its upper part, said annular bulge being intended to be snap-fastened into an annular groove in the stem.

The endless screw may comprise a rounded distal end, having a shape complementary to the end of the stem.

The endless screw is advantageously coupled at its proximal end to a wheel for driving in rotation. The user can turn this wheel manually in order to dispense the composition through the openings in the applicator and load the application surface with the desired quantity of composition.

The endless screw or the driving wheel may comprise a non-return device, for example having a ratchet, so as to avoid rotation of the screw in the opposite direction to the dispensing of the composition.

Applicator

The applicator is intended to be held in the hand and advantageously comprises a gripping member which is disposed in particular on the side of the stem away from the opening(s) and defines a gripping surface.

The longitudinal axis of the applicator is preferably rectilinear. In one variant, the longitudinal axis of the applicator is curvilinear, the applicator having in particular a curved shape at the application surface for the composition.

The applicator may have any shape that gives the applicator surface an ergonomic shape suitable for application onto the desired area, for example the eyelashes and/or eyebrows, the lips or the eyelids.

The stem may have a cross section, taken perpendicularly to its longitudinal axis, with any shape, preferably a circular shape when it serves to guide the endless screw.

The cross section of the stem may be variable along its longitudinal axis. The stem may have a narrowing in its cross section, in particular situated closer to its proximal end than to its distal end, for example at around one third of its length, starting from the proximal end. The narrowing may extend over a distance of between 7 mm and 12 mm. When the stem carries an endless screw that also has a narrowing, the latter advantageously has a shape complementary to that of the stem.

The presence of a widened proximal part makes it possible to increase the volume of the internal space of the stem that is available for containing the composition to be applied. The distal part of smaller diameter makes it possible to reduce the quantity of composition that is driven towards the opening(s) on each turn of the screw and thus to obtain more precise metering.

The inside diameter of the screw, in its proximal portion upstream of the narrowing, may be between 10 mm and 20 mm. The inside diameter of the screw, in its distal portion downstream of the narrowing, may be between 2 mm and 10 mm. When the stem does not have a circular section, the inside diameter is the diameter of the largest circle inscribed in its section.

The stem is preferably made of a thermoplastic material, and is preferably moulded in one piece.

The endless screw may be made of a thermoplastic material. The stem and the endless screw may be made from different materials so as to reduce friction between them.

The length of the stem may be between 9 cm and 15 cm, better still between 10 cm and 12 cm.

Composition

The composition may comprise fibres made of a thermoplastic material, in particular of polyamide. The term “fibres” denotes bodies that are generally elongate, having for example a shape factor ranging from 3.5 to 2500, better still from 5 to 500, in particular from 5 to 150. The shape factor is defined by the ratio LID, where L is the length of the fibre and D the diameter of the circle in which the largest cross section of the fibre is inscribed.

The fibres may have a length of between 0.5 and 3 mm, better still between 1.2 and 2.1 mm, and have a diameter in particular of between 0.5 dtex and 5 dtex.

The fibres may have a mass ranging for example from 0.15 to 30 denier (mass in g for 9 km of thread), better still from 0.18 to 18 denier.

The mass concentration of fibres in the composition is for example between 80 and 100%, better still between 94% and around 100% by mass.

The fibres may have any shape in cross section, for example circular or non-circular, in particular polygonal.

In one variant, the composition comprises flakes, in particular having a largest dimension of between 0.006 mm and 3 mm, better still between 0.1 mm and 1 mm.

The composition may comprise a binder, in particular with a content by mass ranging from 1% to 10% within the composition. The presence of the binder can cause the fibres or flakes to stick to the application surface and of the treated surface, in particular to the eyelashes. The binder may comprise at least one oil, chosen in particular from the oils dimethiconol and dimethicone.

The composition may comprise all the conventional additives, such as preservatives, in particular chosen from the preservatives caprylyl glycol, phenoxyethanol, and pentylene glycol.

In one variant, the composition is a loose or pressed powder.

When the composition comprises fibres or flakes or is a powder, the ratio between the section of the openings in the applicator and the largest dimension of the solid particles of the composition is advantageously between 0.001 and 2, better still between 0.5 and 3, even better still between 0.1 and 1.

The composition may be a paste with a sufficiently high viscosity not to flow under the effect of its own weight, in particular with a viscosity greater than 50 centipoise, better still than 100 centipoise.

The viscosity may be measured at 1 atm and at 25° C. with the aid of a CONTRAVES TV rotary viscometer fitted with an MS-r4 or MS-r5 measuring bar at a frequency of 60 Hz, the measurement being taken after 10 minutes of rotation of the measuring bar within the composition.

The applicator according to the invention is very particularly suitable for applying fibres and/or flakes to the eyelashes and/or eyebrows. It is also generally suitable for dispensing non-liquid compositions, for example a gel, a cast composition or a composition resulting from an inhomogeneous non-liquid mixture, for example for applying high-viscosity haircare formulations to the hair, high-viscosity gloss and/or gloss comprising flakes to the lips or the cheeks, high-viscosity eyeshadow or nail varnish and/or eyeshadow or nail varnish comprising fibres and/or flakes, or high-viscosity foundation.

The applicator according to the invention is not suitable for applying a composition to the teeth or gums.

Application Device

A further subject of the invention is a device for packaging and applying a first cosmetic, in particular makeup, composition to human keratin materials, comprising an applicator according to the invention and a first container holding the product. The first composition is contained in the applicator and comprises for example fibres as mentioned above.

The gripping member of the applicator may constitute a cap for closing this first container. The latter may be empty and serve to protect the applicator when it is not being used, and to collect the composition which may detach therefrom during transport or storage.

The device according to the invention may comprise a second container containing a second composition, and comprising a second applicator comprising an applicator member that defines an application surface. The second applicator may be secured to the first applicator when the first and second containers are closed.

The second composition is preferably other than a non-liquid composition.

The first and second containers may be secured, when the applicator(s) is/are not being used, by virtue of means for coupling the containers.

The device according to the invention may comprise a double closure member, serving to close the first and second containers at the same time. This closure member advantageously acts as a coupling member between the containers.

Makeup Process

A further subject of the invention is a method for making up human keratin materials with the aid of a packaging and application device according to the invention, wherein use is made of an applicator according to the invention to apply the non-liquid composition to the keratin materials.

When the applicator does not have an endless screw, the user can agitate the applicator in order to dispense the composition.

In the variant in which the applicator comprises an endless screw, the user can turn the latter inside the internal space of the applicator such that the rotation of the endless screw causes the composition to flow through the opening(s) in the applicator.

The makeup method may be a “two-step” method and comprise a step, prior to the application of the non-liquid first composition to the keratin materials, of using the abovementioned second applicator to apply the second composition contained in the second container to the keratin materials intended to receive the first composition.

The method may also be a “three-step” method and comprise an additional step in which the second applicator is used again to apply the second composition to the keratin materials coated with the first composition, the latter having been applied by using the applicator according to the invention, consecutively to the previous application of the second composition.

The second composition may thus be a makeup primer, also known as a “base coat”.

The invention may be better understood from reading the following detailed description of nonlimiting implementation examples thereof, and with reference to the appended drawing, in which:

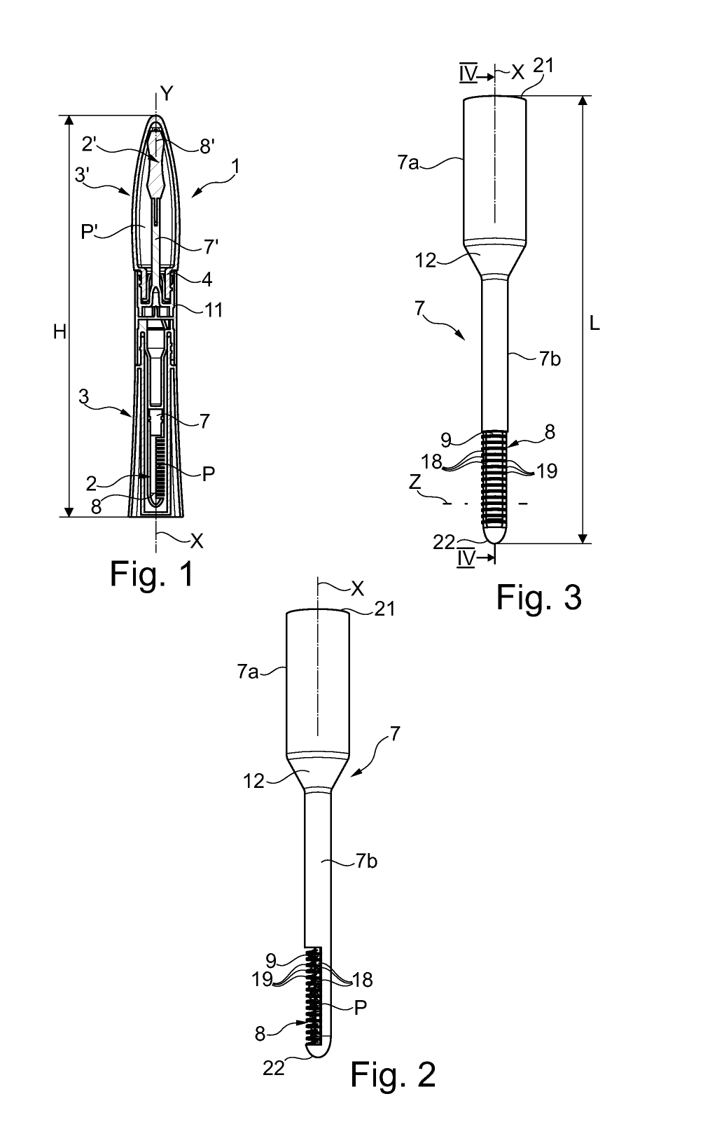

FIG. 1 is a schematic view, in longitudinal section, of an exemplary packaging and application device produced in accordance with the invention,

FIG. 2 shows a side view of the stem of the first applicator from FIG. 1 on its own,

FIG. 3 shows a front view of the stem of the first applicator from FIG. 1 on its own,

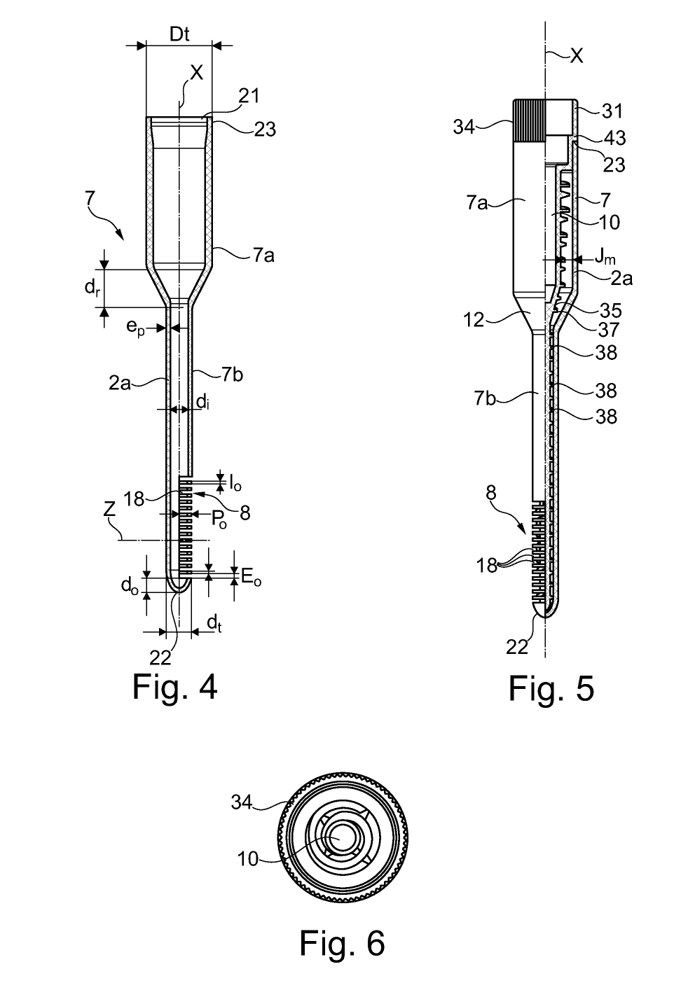

FIG. 4 is an axial section on IV-IV of the stem from FIG. 3,

FIG. 5 is an axial section of a variant embodiment of an applicator according to the invention,

FIG. 6 is a bottom view of the endless screw of the applicator from FIG. 5,

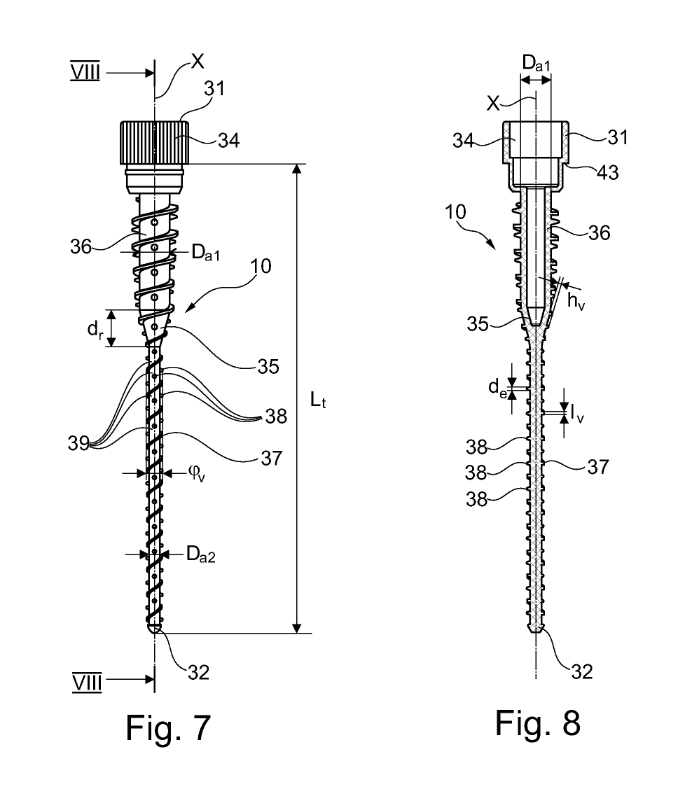

FIG. 7 shows a front view of the endless screw of the applicator from FIG. 5 on its own, and

FIG. 8 is an axial section on VIII-VIII of the screw from FIG. 7.

FIG. 1 shows a packaging and application device 1 produced in accordance with the invention, comprising first 3 and second 3′ containers, and also first 2 and second 2′ associated applicators. The containers 2 and 2′ are disposed in line with one another along the longitudinal axis of the device.

The first applicator 2 is produced in accordance with the invention and comprises, in the example described, a stem 7 that extends along a longitudinal axis X and defines an application surface 8 for the composition P, in the vicinity of its distal end 22.

As can be seen in particular in FIGS. 2 and 3, the applicator 2 defines an internal space 9 that is able to contain a non-liquid composition P to be applied, as defined above.

The second container 3′ comprises a second composition P′ to be applied to the eyelashes, before the first composition.

As illustrated in FIG. 2, the device 1 comprises a double closure member 11 that also acts as a coupling member between the containers 3 and 3′.

In order to use the applicator 2′, the user can leave the container 3 secured to the closure member 11 and thus use the container 3 as a gripping member. Similarly, they can then leave the container 3′ secured to the closure member 11 and use it as a gripping member for using the applicator 2 according to the invention.

In one variant that is not illustrated, the containers 3 and 3′ remain in line with one another and the respective closure members are independent.

In another variant that is not illustrated, the containers 3 and 3′ are situated in line with one another, one of the containers being secured to the closure member of the other container.

In the example in question, the container 3′ comprises a neck 4′ on which the double closure member 11 is arranged in order to be fixed so as to close the container 3′ in a leaktight manner when it is not in use.

The applicator 2′ comprises a stem 7′ of longitudinal axis Y, coincident with the longitudinal axis X of the stem 7 of the first applicator 2, the stem 7′ being connected at its upper end to the double closure member 11 and, at its lower end, to an applicator member that defines an application surface 8′.

Preferably, and as in the example in question, the longitudinal axis Y of the stem 7′ is rectilinear and coincident with the longitudinal axis of the container 3′ when the applicator 2′ is in place thereon, but if the stem 7′ is not rectilinear, forming for example an elbow, this does not depart from the scope of the present invention.

The total height H of the device 1 may be between 17 cm and 25 cm, and is preferably equal to 22 cm.

The length L of the stem 7 may be between 9 cm and 15 cm, better still between 10 cm and 12 cm, and is for example equal to around 11.5 cm.

In the example in question, and as can be seen in particular in FIG. 4, the internal space 9 is defined in the body of the stem 7, along the entire length L thereof, and extends as far as the application surface 8.

The thickness eₚ of the wall 2a of the applicator 2 defining the internal space 9 may be between 0.5 mm and 3 mm, and is for example equal to 1 mm.

In the example described, the stem 7 has a cross section, taken perpendicularly to its longitudinal axis X, having a circular shape.

Preferably, the cross section of the stem 7 is variable along its longitudinal axis X, the stem 7 having, as can be seen in FIGS. 2 to 4, a narrowing 12, situated closer to its proximal end 21 than to its distal end 22, for example at around one third of its length L starting from its proximal end 21. The narrowing 12 may extend over a distance dᵣ of between 7 mm and 12 mm, for example around 9 mm.

As shown in FIG. 4, the diameter Dₜ of the stem 7, in its proximal portion 7a upstream of the narrowing 12, may be between 10 mm and 20 mm, and is for example equal to around 16 mm. The diameter Dₜ of the stem 7, in its distal portion 7b downstream of the narrowing 12, may be between 2 mm and 10 mm, and is for example equal to around 6.2 mm.

As shown in FIGS. 2 to 4, the applicator 2 according to the invention advantageously has a plurality of openings 18 that communicate with the internal space 9 and open onto the application surface 8 so as to allow the composition P to be dispensed and applied.

Preferably, and as in the example in question, the openings 18 are slots that define notches 19, each extending along an axis Z perpendicular to the longitudinal axis X of the stem 7.

The openings 18 are formed, in the example described, over an angular sector equal to 180° around the longitudinal axis X of the stein 7. The openings 18 thus extend to half the thickness of the applicator 2, when seen in side view, and across the entire width of the applicator 2, when seen in front view, as can be seen in FIGS. 2 and 3, respectively.

In one variant that is not illustrated, the openings 18 are formed over an angular sector of less than 180° around the longitudinal axis X of the stem 7.

The most distal 20 opening 18 may extend at a non-zero distance d₀ from the distal end 22 of the stem 7, for example equal to 3.5 mm.

The spacing Eₒ between the openings 18 is for example equal to 0.55 mm.

The width lₒ of the openings 18 is for example equal to 1 mm.

The depth Pₒ of the openings 18 into the wall 2a of the applicator 2 is for example equal to 3 mm.

As in the example in question, the composition P preferably comprises fibres, and the ratio between the section of an opening 18 in the applicator 2 and the length of the fibres is between 0.5 and 3.

In the variant shown in FIGS. 5 to 8, the applicator 2 comprises an endless screw 10, which is carried by the stem 7 and turns inside the internal space 9 such that the rotation of the screw causes the composition P to flow through the openings 18.

The endless screw 10 advantageously extends along the same rectilinear longitudinal axis X as the stem 7, along approximately the entire length L thereof.

In the example in question, the endless screw 10 comprises, as can be seen in FIGS. 4 and 5, an annular bulge 23 designed to be fixed in a corresponding groove in the stem 7.

The endless screw 10 is advantageously coupled at its proximal end 31 to a wheel 34 for driving the screw 10 in rotation in the internal space 9. The user can turn this wheel 34 manually so that the composition P is dispensed through the openings 18 in the applicator 2.

Preferably, as illustrated, the endless screw 10 has a narrowing 35 that is positioned more or less at the narrowing 12 of the stem 7.

In the example in question, the thread 37 of the endless screw 10 is continuous.

The width lᵥ of the thread 37 of the endless screw 10 is for example equal to 0.8 mm.

Along the entire longitudinal axis X of the stem 7, the diameter φᵥ of the endless screw 10, measured at the top of the thread 37, is advantageously strictly less than the inside diameter dᵢ of the facing portion of stem 7.

The clearance Jₘ between the stem 7 and the endless screw 10 may be between 0.1 mm and 4 mm. This clearance Jₘ may be variable along the longitudinal axis X of the stem 7, reaching a maximum at the proximal portion 7a of the stem 7 situated upstream of the narrowing 12, starting from the proximal end 21. This makes it possible to define a larger space for containing the composition P.

The diameter Da₁ of the body 36 of the endless screw 10, in the proximal portion 7a of the stem 7, is for example equal to 7.2 mm. The diameter Da₂ of the body 36 of the endless screw 10, in the distal portion 7b of the stem 7, is for example equal to 2.5 mm.

As can be seen in particular in FIG. 7, the endless screw 10 advantageously comprises protruding elements 38 that are connected to its body 36, along grooves 39 formed by its thread 37. These protruding elements 38 facilitate the dispensing of the composition P, by encouraging the fragmentation thereof. The protruding elements 38 are conical spikes in the example in question.

The endless screw 10 may comprise between 1 and 10 protruding elements 38 per turn.

The height hᵥ of the thread 37 and the protruding elements 38 of the endless screw 10 is for example equal to 0.85 mm.

The largest transverse dimension dₑ of the protruding elements 38 of the endless screw 10, measured at their base, is for example equal to 0.9 mm.

The body 36 of the endless screw 10 is, in the example in question, hollow in its proximal portion.

As shown in FIGS. 7 and 8, the endless screw 10 may have a rounded distal end 32.

In order to mould the stem 7, use can be made of any thermoplastic material which is or is not relatively rigid, for example SEBS, a silicone, latex, a material having improved slip, butyl, EPDM, a nitrile, a thermoplastic elastomer, a polyester elastomer, a polyamide elastomer, a polyethylene elastomer or a vinyl elastomer, a polyolefin such as PE or PP, PVC, EVA, PS, SEBS, SIS, PET, POM, PU, SAM, PA or PMMA.

Preferably, the stem 7 is moulded in one piece.

The endless screw 10 may be made of a polyolefin such as PE or PP, of the polyethylene LDPE, or of POM, PA or ABS.

The user can use the device 1 to apply, during a prior step, the second composition P′, for example mascara, to the eyelashes, with the aid of the second applicator 2′. They can then use the applicator 2 according to the invention to apply the composition P to the keratin materials.

When the applicator 2 does not have an endless screw 10, the user can agitate the applicator 2 in order to dispense the composition P. In the variant in which the applicator 2 comprises an endless screw 10, the user turns the latter such that the rotation causes the composition P to flow through the openings 18.

During an additional step, the user can reuse the second applicator 2′ in order to reapply the second composition P′, in order to perfect the makeup.

Of course, the invention is not limited to the exemplary embodiments that have just been described. In particular, the invention can also be applied to makeup for the skin, nails, lips or hair.

The expression “having a” should be understood as being synonymous with “having at least one”, and “between” is understood as including the limits, unless specified to the contrary.

1. Cosmetic applicator for applying a non-liquid composition to human keratin materials, the applicator:
comprising a stem that extends along a longitudinal axis,
defining an internal space that contains the non-liquid composition to be applied, and
having at least one opening that is in communication with the internal space and leads onto an application surface.

2. Cosmetic applicator for applying a composition to human keratin materials, the applicator:
comprising a stem that extends along a longitudinal axis and bears an endless screw,
defining an internal space that is able to contain the composition to be applied, and
having at least one opening that is in communication with the internal space in order to dispense said composition,
wherein the endless screw turns inside the internal space such that its rotation causes the composition to flow through the opening(s).

3. Applicator according to claim 1, which has a plurality of openings in the form of slots, in particular between 5 and 50 openings, better still between 10 and 20 openings.

4. Applicator according to claim 2, wherein the endless screw comprises a body and protruding elements that are connected thereto and make it easier to dispense the composition.

5. Applicator according to claim 4, wherein the protruding elements on the endless crew follow the same helical path as the thread thereof.

6. Applicator according to claim 4, wherein the protruding elements on the endless crew are the same height as the thread thereof, being in particular between 0.5 mm and 1.5 mm, better still between 0.7 mm and 1.2 mm.

7. Applicator according to claim 4, in which the clearance between the stem and the thread of the endless screw is between 0.1 mm and 4 mm, better still between 0.5 mm and 3 mm.

8. Applicator according to claim 1, wherein the composition comprises fibres made of a thermoplastic material, in particular polyamide, with a length in particular of between 0.5 and 3 mm, better still between 1.2 and 2.1 mm, and with a diameter in particular of between 0.5 dtex and 5 dtex.

9. Applicator according to claim 2, wherein the composition comprises flakes, in particular with a largest dimension of between 0.006 mm and 3 mm.

10. Applicator according to claim 1, wherein the composition is a loose or pressed powder.

11. Applicator according to claim 1, wherein, with the composition comprising fibres or flakes or being a powder, the ratio between the section of the openings in the applicator and the largest dimension of the solid particles of the composition is between 0.001 and 2, better still between 0.5 and 3.

12. Applicator according to claim 1, wherein the composition comprises a binder, in particular with a content by mass ranging from 1% to 10% within the composition, comprising in particular at least one oil.

13. Applicator according to claim 1, wherein the composition is a paste with a sufficiently high viscosity not to flow under the effect of its own weight.

14. Device for packaging and applying a first cosmetic composition, in particular a makeup composition, to human keratin materials, comprising an applicator according to claim 1 and a first container holding the applicator.

15. Device according to claim 14, which comprises a second container containing a second composition, and comprises a second applicator comprising an applicator member that defines an application surface, the second applicator being in particular secured to the first applicator, the second composition being in particular other than a non-liquid composition.

16. Method for making up or caring for human keratin materials with the aid of a packaging and application device according to claim 14, wherein use is made of an applicator to apply the non-liquid composition to the keratin materials, the applicator:
comprising a stem that extends along a longitudinal axis,
defining an internal space that contains the non-liquid composition to be applied, and
having at least one opening that is in communication with the internal space and leads onto an application surface.

17. Method according to claim 16, wherein the endless screw is turned inside the internal space of the applicator such that the rotation of the endless screw causes the composition to flow through the opening(s) in the applicator.

18. Method according to claim 16, which comprises a step prior to the application of the non-liquid first composition to the keratin materials, of using the second applicator to apply the second composition contained in the second container to the keratin materials intended to receive the first composition.
